(19) United States
(12) Patent Application Publication
(10) Pub. No.: US 2024/0194358 A1
(43) Pub. Date: Jun. 13, 2024

(54) SYSTEMS AND METHODS FOR AUTOMATED PRICING, CONDUCTION, AND TRANSCRIPTION OF TELEMEDICINE ENCOUNTERS

(71) Applicant: Remote Health Ventures LLC, New York, NY (US)

(72) Inventors: Riley Williams, III, New York, NY (US); Benedict Nwachukwu, New York, NY (US)

(73) Assignee: Remote Health Ventures LLC, New York, NY (US)

(21) Appl. No.: 18/427,780

(22) Filed: Jan. 30, 2024

Related U.S. Application Data
(62) Division of application No. 17/239,939, filed on Apr. 26, 2021.
(60) Provisional application No. 62/704,160, filed on Apr. 24, 2020.
(60) Provisional application No. 63/015,256, filed on Apr. 24, 2020.
(60) Provisional application No. 62/706,139, filed on Aug. 3, 2020.
(60) Provisional application No. 62/706,142, filed on Aug. 3, 2020.

(51) Int. Cl. G16H 80/00; G16H 10/60; G16H 40/20

(52) U.S. Cl. CPC G16H 80/00; G16H 10/60; G16H 40/20

(57) ABSTRACT

Various systems and methods are described herein that facilitate the automatic monitoring, evaluating, and analysis of information gathered during a telemedicine appointment. The systems and methods are further adapted to generate automated clinical notes. Such systems, methods and apparatuses further allow for flexible, dynamic pricing and scheduling of telemedicine services based on a number of different factors including patient preferences and physician characteristics.

### CROSS-REFERENCE TO RELATED APPLICATIONS

This application is a divisional of U.S. Non-Provisional application Ser. No. 17/239,939, filed Apr. 26, 2021 and entitled “SYSTEMS AND METHODS FOR AUTOMATED PRICING, CONDUCTION, AND TRANSCRIPTION OF TELEMEDICINE ENCOUNTERS”, which claims priority to U.S. Provisional Patent Application No. 62/704,160, filed Apr. 24, 2020 and entitled “APPLICATION OF ARTIFICIAL INTELLIGENCE FOR THE MUSCULOSKELETAL PHYSICAL EXAMINATION AND KINEMATIC CAPTURE DURING TELEMEDICINE ENCOUNTER”, U.S. Provisional Patent Application No. 63/015,256, filed Apr. 24, 2020 and entitled “APPLICATION OF ARTIFICIAL INTELLIGENCE FOR AUTOMATED SPEECH RECOGNITION DURING A TELEMEDICINE ENCOUNTER”, U.S. Provisional Patent Application No. 62/706,139, filed Aug. 3, 2020 and entitled “SYSTEMS AND METHODS FOR CREATING DYNAMIC PRICING ALGORITHMS FOR A VIRTUAL ENCOUNTER,” and U.S. Provisional Patent Application No. 62/706,142, filed Aug. 3, 2020, and entitled “SYSTEMS AND METHODS FOR MATCHING PATIENTS AND DOCTORS FOR AN ON-DEMAND VIRTUAL ENCOUNTER.” Each of the above applications is incorporated by reference herein in its entirety.

### BACKGROUND

This specification relates generally to systems and methods for aiding healthcare providers in rendering care remotely to patients. Technological advances in videoconferencing, software, and digital applications have enabled a transition to virtual care delivery. Specifically, “telemedicine” or “telehealth” involves the provision of health-related services using audiovisual, electronic, and telecommunication tools. Telemedicine allows for remote consultation between a patient and a provider, and include services such as diagnosis, monitoring, counseling, and rehabilitation.

While telemedicine has made significant progress in simulating in-person healthcare appointments, it is currently limited by providers' inability to perform physical examinations on patients.

Furthermore, upon meeting with a patient, providers are generally required to document a summary regarding the appointment in the form of a clinical note or other form of written document, as well as the provider's assessment and recommendation. Such clinical notes take up a significant portion of a physicians' day, as a recent study has shown that they generally spend on average over 16 minutes on such notes for each patient visit.

Rigid, outdated payment models are another limitation of the current telemedicine system. Currently available payment models result in a less satisfactory patient experience due to the lack of choice afforded to patients in choosing their providers. For example, in such models, a predetermined set rate is applied to patient-physician encounters, and the “service” fee is set by the third-party insurer, service provider, and in some cases, the provider.

Yet another impediment to ubiquitous, successful implementation of telemedicine is the lack of automated scheduling systems for scheduling on-demand telemedicine appointments in a time-sensitive and transparent manner. Such scheduling systems further fail to reflect patient preferences for their providers.

Accordingly, there is a need for systems and methods that facilitate the collection and analysis of various patient information during remote physical examination of said patients. It would also be beneficial if such systems and methods could quickly and accurately generate automated clinical notes for providers during remote telemedicine appointments.

It would be further beneficial if such systems and methods were adapted to utilize a dynamic, transparent pricing algorithm taking into account such factors as patient preferences, provider characteristics, and industry factors such as the supply and demand of providers to schedule such telemedicine appointments and set consultation fees. It would also be helpful if such systems and methods facilitated the scheduling of on-demand appointments to match patients' time-sensitive needs for care.

### SUMMARY

In accordance with the foregoing objectives and others, exemplary systems and methods are disclosed herein for facilitating the scheduling, pricing, and execution of telemedicine appointments. The disclosed systems and methods may allow for more efficient, accurate extraction and analysis of patient and medical examination information during the telemedicine appointments via speakers and cameras from wireless communication technologies that transmit dynamic audio and visual data. The disclosed systems and methods may further allow for audio data received during the telemedicine appointments to be interpreted and automatically transcribed into clinical notes through automatic speech recognition algorithms.

The disclosed systems and methods may also be adapted to utilize a dynamic pricing system that updates in real time and accounts for patient preferences, physician characteristics, and industry forces. The disclosed systems and methods may further allow for automatic, efficient, and seamless scheduling of on-demand telemedicine appointments.

In one embodiment, a computer-implemented method for the collection and analysis of information received during a telemedicine appointment is provided. The method may include receiving, from a patient device, patient information and one or more physical examination parameters. The method may also include determining, based on one or more of the received patient information and the received physical examination parameters, a patient guideline. The method may further include transmitting, to the patient device, the patient guideline. The method may include receiving, from the patient device, feedback information. The method may include processing and storing the feedback information. The processing of the feedback information may include filtering the feedback information, classifying the feedback information, quantifying the feedback information, and associating the feedback information with a session. The method may also include determining a second patient guideline based on one or more of the patient information, the received physical examination parameters, and the feedback information. The method may include receiving, from the patient device, second feedback information. The method may also include processing and storing the second feedback information. The method may further include associating the second feedback information with the session. Such session information may include patient information, feedback information, and second feedback information. The method may also include determining that the session is completed. The method may include determining a treatment recommendation based on the session information.

In another embodiment, a computer-implemented method for automatic speech and speaker recognition and transcription is provided. The method may include displaying, on a patient device, a question. The method may also include receiving audio data corresponding to speech. Such audio data may include a plurality of audio frames. The method may include performing automatic speech recognition on the plurality of audio frames to determine text data corresponding to a transcript. The method may also include determining a user profile associated with the patient device. The method may include transmitting the transcript to a provider device. The method may also include receiving transcription feedback information from the provider device. The transcription feedback information may include a score representing the degree of similarity between the audio data and the transcript

In another embodiment, a computer-implemented method for scheduling and dynamically pricing a telemedicine appointment is provided. The method may include storing provider information associated with one or more providers. The method may also include receiving a request for a telemedicine appointment from a patient. The request may include patient information and patient preferences information associated with the patient. The patient preferences information may further include: a budget range for the telemedicine appointment, a preference ranking associated with the budget range, a location of the telemedicine encounter, and a preference ranking associated with location. The method may also include determining, based on one or more of patient information, the provider information, and the patient preferences information, a matching provider and a fee associated with the matching provider. The method may include transmitting matching provider information associated with the matching provider to the patient. The matching provider information may include a plurality of available appointment times and the fee. The method may include receiving a selection of the matching provider and an available appointment time. The method may include transmitting the fee to the patient. The method may include receiving the fee. The method may include transmitting a confirmation to the patient. Said confirmation may include the name of the selected matching provider, the selected appointment time, and the received fec. In another embodiment, a method for scheduling an on-demand telemedicine appointment is provided. The method may include storing provider information associated with one or more providers. The method may also include receiving a request for an on-demand telemedicine appointment from a patient. Said request may include patient information and patient preferences information associated with the patient. The patient preferences information may further include an indication of the degree of urgency of the request and a location. The method may include determining, based on the patient preferences information and the provider information, a plurality of matching providers. The method may further include transmitting, to the patient, at least a portion of the provider information associated with each of the plurality of matching providers. The provider information may include one or more available appointment times and a location. The method may also include receiving a selection of two matching providers from the plurality of matching providers. The first matching provider may be associated with a first preference ranking, and the second matching provider is associated with a second preference ranking. The method may include transmitting a fee to the patient. This fee may be associated with the first matching provider. The method may include receiving the fee. The method may include transmitting a notification to the first matching provider. Said notification may include appointment information further including patient information and the appointment time and a request to confirm the appointment time. The method may include determining that the first matching provider is not available. The method may include transmitting a notification to the second matching provider. Said notification may include appointment information further including patient information and the appointment time and a request to confirm the appointment time. The method may also include receiving a confirmation, by the second matching provider, to the notification. The method may include transmitting the confirmation to the patient. The confirmation may include the name of the second matching provider, the appointment time, and the received fec.

### BRIEF DESCRIPTION

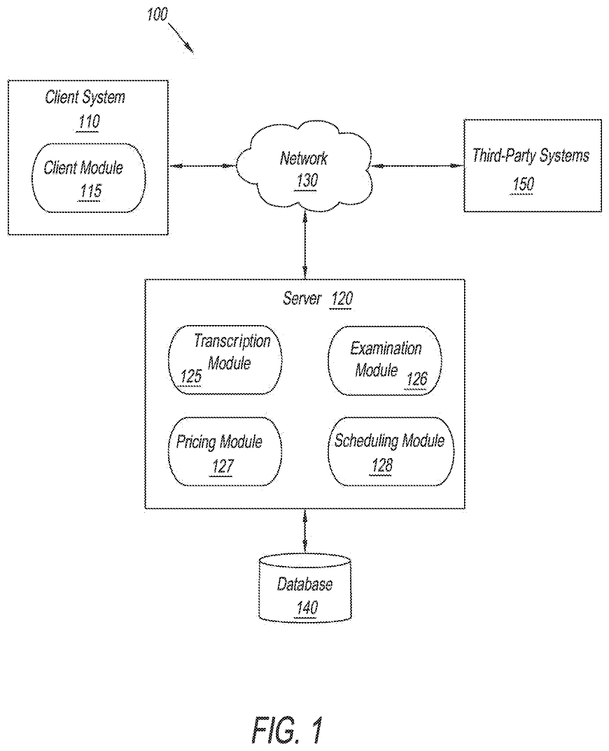

FIG. 1 shows an exemplary telemedicine system 100 in accordance with one or more embodiments presented herein.

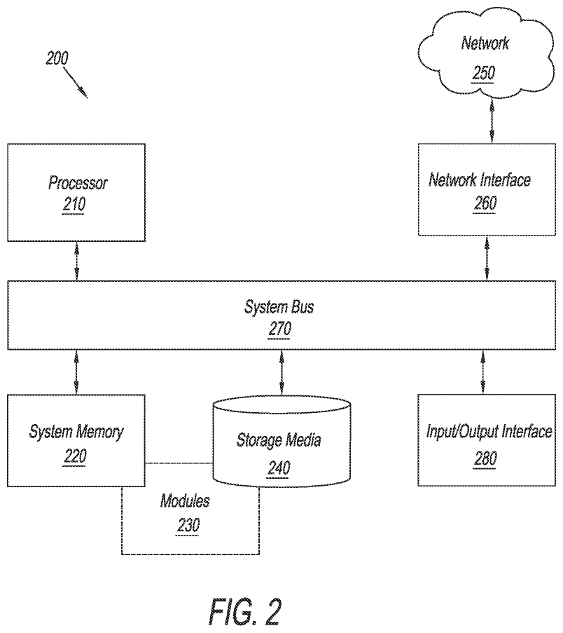

FIG. 2 shows an exemplary computing machine 200 and modules 230 in accordance with one or more embodiments presented herein.

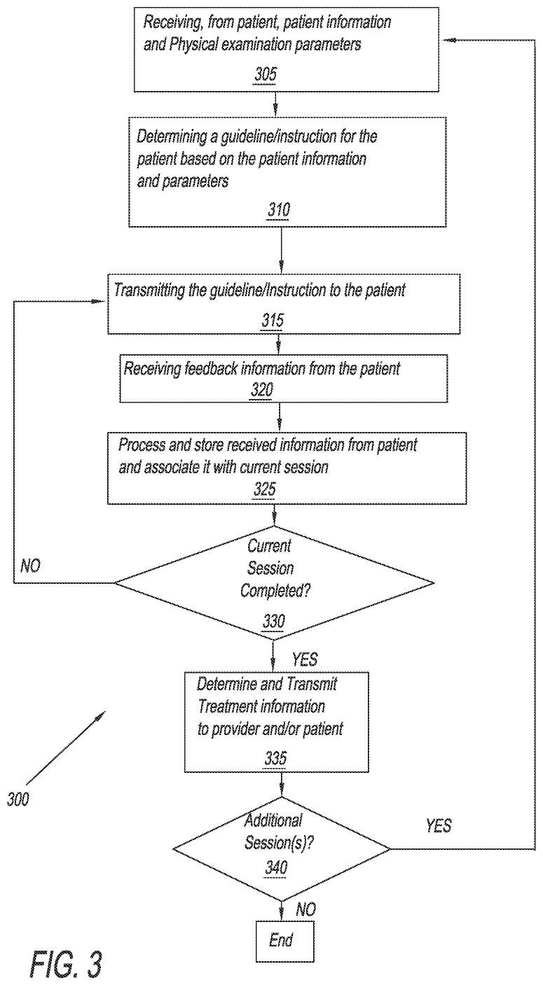

FIG. 3 shows a method 300 for conducting a remote inspection/physical examination in accordance with one or more embodiments presented herein.

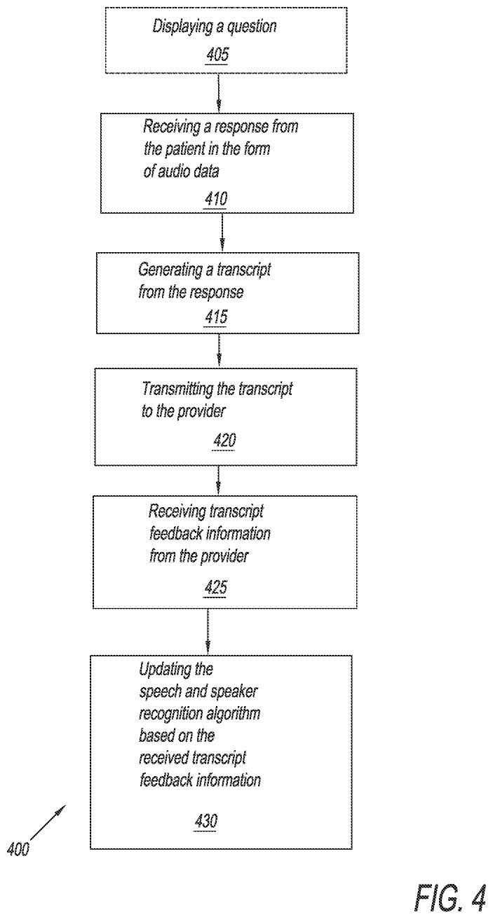

FIG. 4 shows a method 400 for generating a transcript from spoken words in accordance with one or more embodiments presented herein.

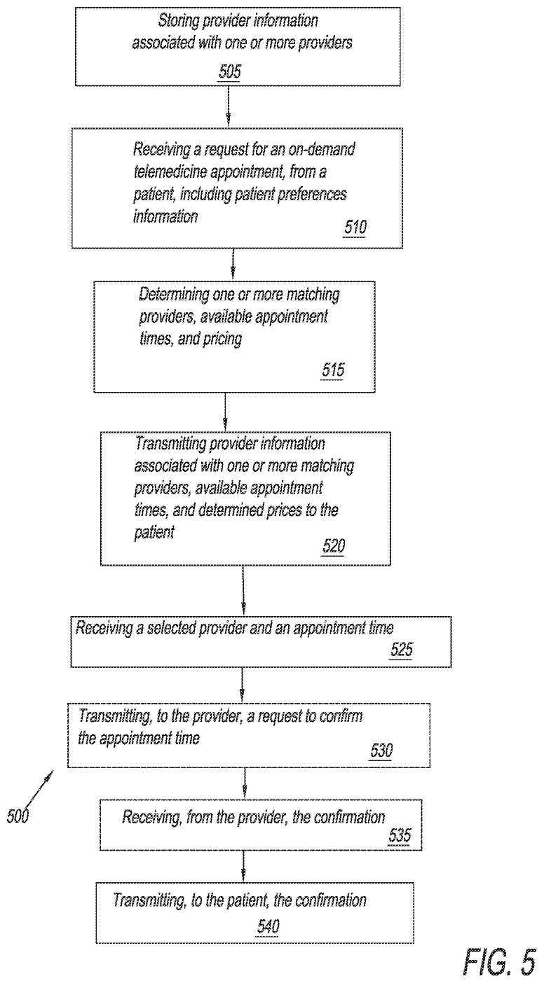

FIG. 5 shows a method 500 for dynamically pricing a telemedicine appointment in accordance with one or more embodiments described herein.

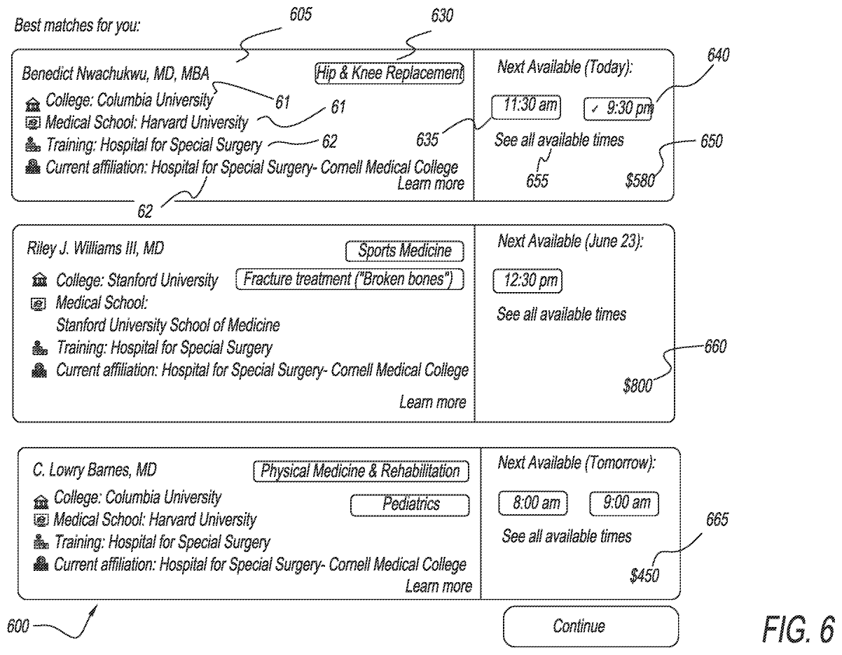

FIG. 6 shows a matched providers screen 600 displaying one or more matched providers in accordance with one or more embodiments described herein.

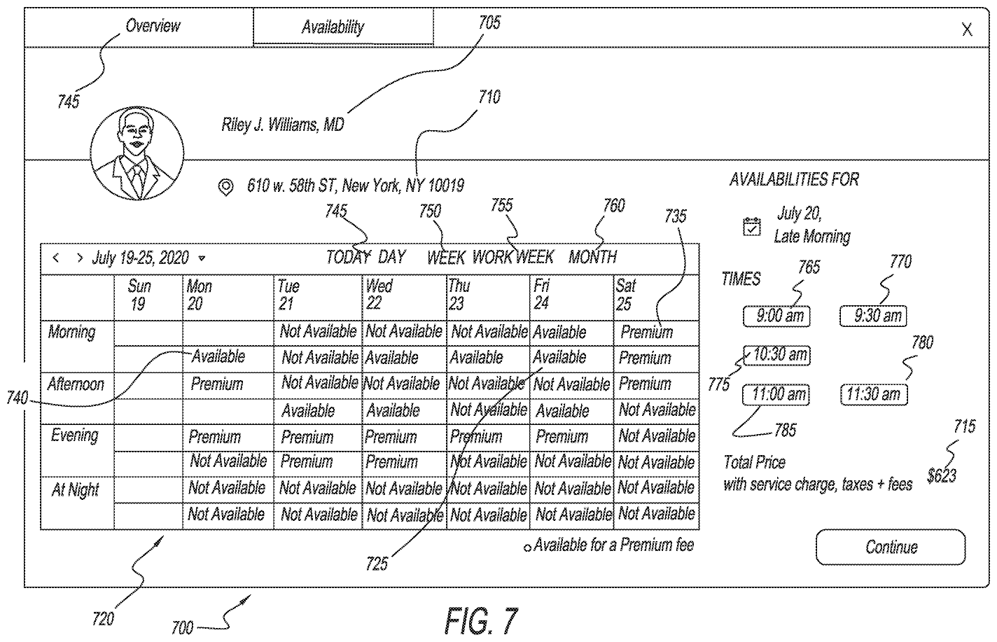

FIG. 7 shows a provider availability details screen 700 in accordance with one or more embodiments described herein.

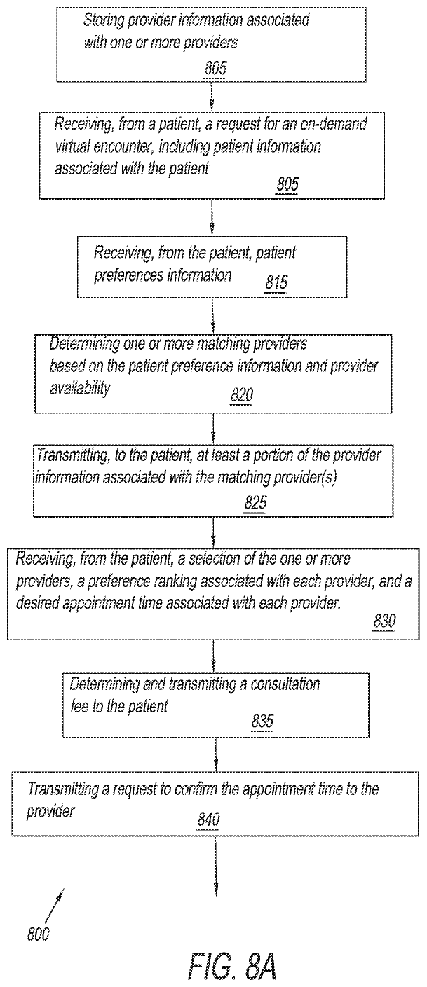

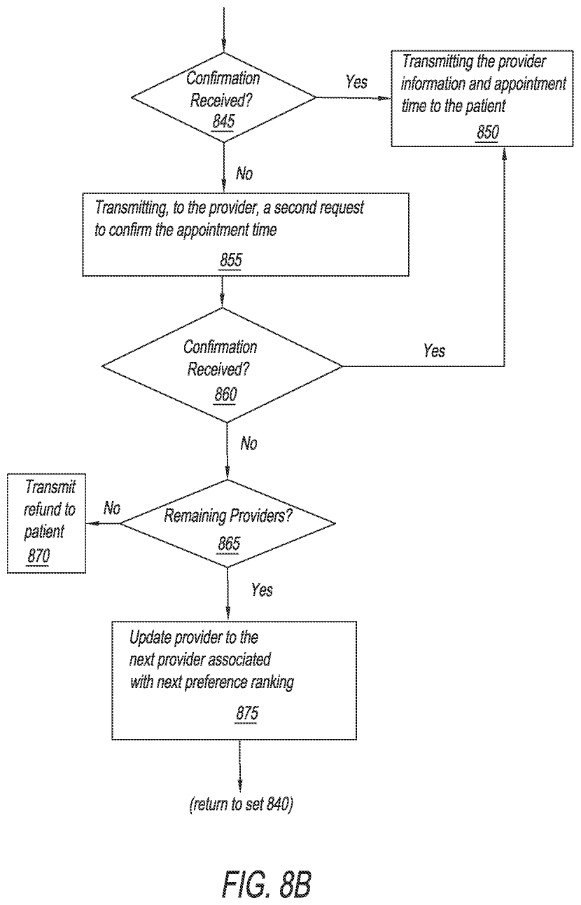

FIG. 8 shows a method 800 scheduling on-demand telemedicine appointments in accordance with one or more embodiments described herein.

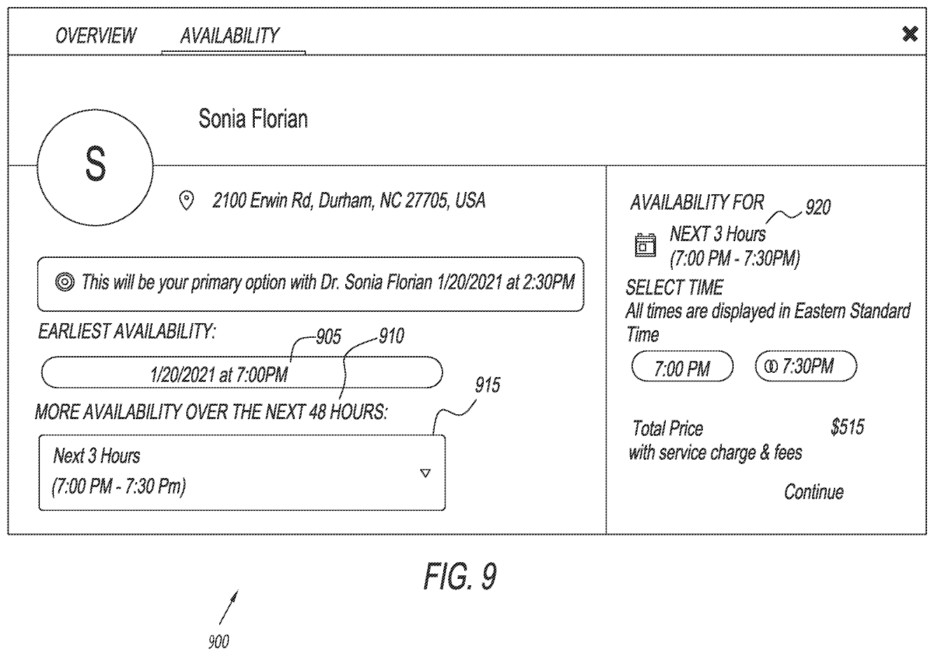

FIG. 9 shows a a provider availability details screen 900 in accordance with one or more embodiments described herein.

### DETAILED DESCRIPTION

Various systems and methods are disclosed herein that facilitate the scheduling, pricing, and execution of telemedicine appointments. The disclosed embodiments improve upon currently available tools for monitoring and evaluating patients via remote medical physical examinations and inspections. Specifically, the disclosed embodiments allow for such examinations to be conducted remotely through speakers, cameras, and other sensors which utilize wireless communication to transmit dynamic audio and visual data during telemedicine appointments that typically rely on human interpretation to draw meaningful conclusions.

The disclosed embodiments may also facilitate the transcription of clinical notes during the remote appointments. Generally, as the patient interacts with the system during the appointment, the speech recognition software may perform both voice recognition and speaker identification while simultaneously capturing components of the speaker's speech and language. The system may then transcribe these components into a clinical note or other summative document.

Furthermore, the disclosed embodiments may facilitate the scheduling of telemedicine appointments by matching patients to providers in accordance with patient preferences, such as a preference for a specific location, a certain price range, or immediate care/care within a certain period of time. Such embodiments may further optimize the pricing of such appointments by taking into account the patient preferences along with provider availability and other provider characteristics (e.g., reputation, specialty, etc.). In embodiments where patients may request an on-demand telemedicine appointment, the disclosed embodiments may immediately determine one or more matching providers, then notify and contact the providers selected by the patient in a way such that the patient's preferences and desire for rendered care within a certain time is fully optimized.

Referring to FIG. 1, a block diagram depicting a telemedicine system 100 in accordance with one or more embodiments is illustrated.

As shown, the system may comprise one or more client devices and/or client systems 110 interfacing with a server 120 that transmits and/or receives data to/from a database 140. Each of the client system 110, the server 120 and the database 140 may communicate over one or more networks (e.g., network 130).

As detailed below in reference to FIG. 2, the server 120 may comprise any number of computing machines and associated hardware/software, where each computing machine may be located at one site or distributed across multiple sites and interconnected by a communication network. The server 120 may provide the backend functionality of the telemedicine system 100.

To that end, the server 120 may execute a telemedicine application comprising various modules, such as a transcription module 125, an examination module 126, a pricing module 127, and a scheduling module 128. The telemedicine application may be adapted to present various user interfaces to users, where such interfaces may be based on information stored on a client system 110 and/or received from the server 120. The telemedicine application may be written in any form of programming language, including compiled or interpreted languages, or declarative or procedural languages, and it can be deployed in any form, including as a standalone program or as a module, component, subroutine, or other unit suitable for use in a computing environment. Such software may correspond to a file in a file system. A program can be stored in a portion of a file that holds other programs or data. For example, a program may include one or more scripts stored in a markup language document; in a single file dedicated to the program in question; or in multiple coordinated files (e.g., files that store one or more modules, sub programs, or portions of code).

Generally, client systems 110 may comprise any systems or devices capable of running a client module 115 and/or of accessing the server 120. As discussed below in reference to FIG. 2, exemplary client systems 110 may comprise computing machines, such as general purpose desktop computers, laptop computers, tablets, smartphones wearable devices, virtual reality (“VR”) devices and/or augmented reality (“AR”) devices.

The client module 115 may be adapted to communicate with the telemedicine application and/or the various modules running on the server 120. Exemplary client modules 115 may comprise a computer application, a native mobile application, a webapp, or software/firmware associated with a kiosk, set-top box, or other embedded computing machine. In one embodiment, a user may download an application comprising a client module 115 to a client system (e.g., from the Google Play Store or Apple App Store). In another embodiment, a user may navigate to a webapp comprising a client module 115 using an internet browser installed on a client system 110. In certain embodiments known as “thin clients”, the client module 115 may be located on the server 120 (rather than on the client system) and the client system 110 will communicate with the client module 115 on the server 120 via the network 130.

The server 120 and client systems 110 may be adapted to receive and/or transmit application information to/from various users (e.g., via any of the above-listed modules). Such systems may be further adapted to store and/or retrieve application information to/from one or more local or remote databases (e.g., database 140). Exemplary databases 140 may store received data in one or more tables, such as but not limited to, a patients table, health providers table, appointments table, and/or others.

Optionally, the telemedicine system 100 may additionally comprise any number of third-party systems 150 connected to the server 120 via the network 130. Third-party systems 150 typically store information in one or more remote databases that may be accessed by the server 120. Third-party systems may include, but are not limited to: transcriptions systems, payment systems, location and navigation systems, backup systems, communication systems and/or others. The server 120 may be capable of retrieving and/or storing information from third-party systems 150, with or without user interaction. Moreover, the server 120 may be capable of communicating information (e.g., information stored in the database 140) to any number of third-party systems and may notify users of such communications.

Referring to FIG. 2, block diagram is provided illustrating a computing machine 200 and modules 230 in accordance with one or more embodiments presented herein. The computing machine 200 may correspond to any of the various computers, servers, mobile devices, embedded systems, or computing systems discussed herein. For example, the computing machine 200 may correspond to the client systems 110, server 120 and/or third-party systems 150 shown in FIG. 1.

A computing machine 200 may comprise all kinds of apparatuses, devices, and machines for processing data, including but not limited to, a programmable processor, a computer, and/or multiple processors or computers. As shown, an exemplary computing machine 200 may include various internal and/or attached components such as processor 210, system bus 270, system memory 220, storage media 240, input/output interface 280, and network interface 260 for communicating with a network 250.

The computing machine 200 may be implemented as a conventional computer system, an embedded controller, a laptop, a server, a mobile device, a smartphone, a set-top box, over-the-top content TV (“OTT TV”), Internet Protocol television (“IPTV”), a kiosk, a vehicular information system, one more processors associated with a television, a customized machine, any other hardware platform and/or combinations thereof. Moreover, a computing machine may be embedded in another device, such as but not limited to, a mobile telephone, a personal digital assistant (“PDA”), a smartphone, a tablet, a mobile audio or video player, a game console, a Global Positioning System (“GPS”) receiver, or a portable storage device (e.g., a universal serial bus (“USB”) flash drive). In some embodiments, the computing machine 200 may be a distributed system configured to function using multiple computing machines interconnected via a data network or bus system 270.

The processor 210 may be configured to execute code or instructions to perform the operations and functionality described herein, manage request flow and address mappings, and to perform calculations and generate commands. The processor 210 may be configured to monitor and control the operation of the components in the computing machine 200. The processor 210 may be a general-purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 210 may be a single processing unit, multiple processing units, a single processing core, multiple processing cores, special purpose processing cores, coprocessors, or any combination thereof. In addition to hardware, exemplary apparatuses may comprise code that creates an execution environment for the computer program (e.g., code that constitutes one or more of: processor firmware, a protocol stack, a database management system, an operating system, and a combination thereof). According to certain embodiments, the processor 210 and/or other components of the computing machine 200 may be a virtualized computing machine executing within one or more other computing machines.

The system memory 220 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 220 also may include volatile memories, such as random access memory (“RAM”), static random access memory (“SRAM”), dynamic random access memory (“DRAM”), and synchronous dynamic random access memory (“SDRAM”). Other types of RAM also may be used to implement the system memory. The system memory 220 may be implemented using a single memory module or multiple memory modules. While the system memory is depicted as being part of the computing machine 200, one skilled in the art will recognize that the system memory may be separate from the computing machine without departing from the scope of the subject technology. It should also be appreciated that the system memory may include, or operate in conjunction with, a non-volatile storage device such as the storage media 240.

The storage media 240 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 240 may store one or more operating systems, application programs and program modules such as module, data, or any other information. The storage media may be part of, or connected to, the computing machine 200. The storage media may also be part of one or more other computing machines that are in communication with the computing machine such as servers, database servers, cloud storage, network attached storage, and so forth.

The modules 230 may comprise one or more hardware or software elements configured to facilitate the computing machine 200 in performing the various methods and processing functions presented herein. The modules 230 may include one or more sequences of instructions stored as software or firmware in association with the system memory 220, the storage media 240, or both. The modules 230 may also comprise hardware circuits or information for configuring hardware circuits such as microcode or configuration information for an FPGA or other PLD. Exemplary modules include, but are not limited to, the modules discussed above with respect to FIG. 1 (e.g., the client module 115, transcription module 125, examination module 126, pricing module 127 and scheduling module 128) or any other scripts, web content, software, firmware and/or hardware.

In one embodiment, the storage media 240 may represent examples of machine or computer readable media on which instructions or code may be stored for execution by the processor. Machine or computer readable media may generally refer to any medium or media used to provide instructions to the processor. Such machine or computer readable media associated with the modules may comprise a computer software product. It should be appreciated that a computer software product comprising the modules may also be associated with one or more processes or methods for delivering the module to the computing machine via the network, any signal-bearing medium, or any other communication or delivery technology.

The input/output (“I/O”) interface 280 may be configured to couple to one or more external devices, to receive data from the one or more external devices, and to send data to the one or more external devices. Such external devices along with the various internal devices may also be known as peripheral devices. The I/O interface 280 may include both electrical and physical connections for operably coupling the various peripheral devices to the computing machine 200 or the processor 210. The I/O interface 280 may be configured to communicate data, addresses, and control signals between the peripheral devices, the computing machine, or the processor. The I/O interface 280 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attachment (“ATA”), serial ATA (“SATA”), universal serial bus (“USB”), Thunderbolt, FireWire, various video buses, and the like. The I/O interface may be configured to implement only one interface or bus technology. Alternatively, the I/O interface may be configured to implement multiple interfaces or bus technologies. The I/O interface may be configured as part of, all of, or to operate in conjunction with, the system bus 270. The I/O interface 280 may include one or more buffers for buffering transmissions between one or more external devices, internal devices, the computing machine 200, or the processor 210.

The I/O interface 280 may couple the computing machine 200 to various input devices including mice, touch-screens, scanners, biometric readers, electronic digitizers, sensors, receivers, touchpads, trackballs, cameras, microphones, keyboards, any other pointing devices, or any combinations thereof. When coupled to the computing device, such input devices may receive input from a user in any form, including acoustic, speech, visual, or tactile input.

The I/O interface 280 may couple the computing machine 200 to various output devices such that feedback may be provided to a user via any form of sensory feedback (e.g., visual feedback, auditory feedback, or tactile feedback). For example, a computing device can interact with a user by sending documents to and receiving documents from a device that is used by the user (e.g., by sending web pages to a web browser on a user's client device in response to requests received from the web browser). Exemplary output devices may include, but are not limited to, displays, speakers, printers, projectors, tactile feedback devices, automation control, robotic components, actuators, motors, fans, solenoids, valves, pumps, transmitters, signal emitters, lights, and so forth. And exemplary displays include, but are not limited to, one or more of: projectors, cathode ray tube (“CRT”) monitors, liquid crystal displays (“LCD”), light-emitting diode (“LED”) monitors and/or organic light-emitting diode (“OLED”) monitors.

Embodiments of the subject matter described in this specification can be implemented in a computing machine 200 that includes one or more of the following components: a backend component (e.g., a data server); a middleware component (e.g., an application server); a frontend component (e.g., a client computer having a graphical user interface (“GUI”) and/or a web browser through which a user can interact with an implementation of the subject matter described in this specification); and/or combinations thereof. The components of the system can be interconnected by any form or medium of digital data communication, such as but not limited to, a communication network.

Accordingly, the computing machine 200 may operate in a networked environment using logical connections through the network interface 260 to one or more other systems or computing machines 200 across the network 250. The network 250 may include wide area networks (“WAN”), local area networks (“LAN”), intranets, the Internet, wireless access networks, wired networks, mobile networks, telephone networks, optical networks, or combinations thereof. The network 250 may be packet switched, circuit switched, of any topology, and may use any communication protocol. Communication links within the network 250 may involve various digital or an analog communication media such as fiber optic cables, free-space optics, waveguides, electrical conductors, wireless links, antennas, radio-frequency communications, and so forth.

The processor 210 may be connected to the other elements of the computing machine 200 or the various peripherals discussed herein through the system bus 270. It should be appreciated that the system bus 270 may be within the processor, outside the processor, or both. According to some embodiments, any of the processor 210, the other elements of the computing machine 200, or the various peripherals discussed herein may be integrated into a single device such as a system on chip (“SOC”), system on package (“SOP”), or ASIC device.

Referring to FIG. 3, a method 300 for automatically collecting, analyzing, and transmitting information during a remote inspection/physical examination is illustrated. The exemplary embodiments generally allow for inspections and physical examinations to be conducted remotely using speakers, cameras, and other sensors which utilize wireless communication to inspect, track, and transmit dynamic audio and visual data during telemedicine appointments that typically rely on human interpretation to draw meaningful conclusions. The system may automatically interpret such data without requisite human interpretation through the use of machine learning and other advanced, artificial intelligence-based analytic processes.

The system may continuously monitor the patient's movement from or during the telemedicine appointment, evaluating and tracking qualitative and quantitative metrics, such as the current exam maneuver being performed, the number of repetitions of a rehabilitation exercise, or the range of motion of the affected musculoskeletal unit, all in real time and/or after the user has performed the routine. Thus, the patient is not required to provide any input and is free to participate in the telemedicine encounter without interruption of inputting data. Surgeons, therapists, or other healthcare providers engaged in performing a musculoskeletal physical exam remotely during a telemedicine encounter may use the application for remote patient monitoring.

Generally, the telemedicine appointment may consist of an initial inspection of the joint or body region of interest. At step 305, the system receives, from a patient, patient information and parameters for the physical examination. Exemplary patient information may include: identification information (e.g., name, age, date of birth, sex, social security number, unique ID, photo, etc.); consent information (e.g., scanned consent forms and/or recorded dates of consent forms for use of the provider device and/or for sharing medical information relating to the use thereof); billing information (e.g., credit card information, billing address, etc.); insurance information (e.g., insurance provider, plan, type, benefits, member number, group number, etc.); and/or current and/or historical medical information (e.g., illnesses, injuries, types of medications taken, dosage to be taken, days and/or times when such medication should be taken, physical therapy routines, radiological studies, reported symptoms etc.). Patient information may also include information relating to third parties associated with the patient (e.g., a doctor or other health care provider who may conduct a pain measurement session and/or users who may access patient information associated with the patient). Parameters for the inspection may include: gait, joint symmetry, loss of contour, atrophy, and deformation.

At step 310, the system determines a guideline/instruction for the patient based on the received patient information and parameters. Generally, patients will receive one or more inspection guidelines and/or instructions relating to an initial inspection conducted by the system. The initial inspection guideline generally pertains to a joint or body region of interest, which may include the entire spine, shoulder, elbow, wrist, hip, knee, and/or ankle.

While in the described embodiment, said guidelines and/or instructions may be determined and provided by system automatically, in other embodiments, the instructions may be determined by the provider, lead by another instructor, or by an animated or computer generated sample or model. For example, in one embodiment, the exam maneuvers may be planned and suggested by healthcare providers or automated software cue. For example, an orthopedic surgeon may specify a particular physical exam routine during the telemedicine appointment and the user may follow the suggested exam routine. The provider may perform a portion of the routine, or recite certain instructions, which may be recorded by the system using cameras, microphones, motion capture systems, or the like; to be used as instruction. The system and/or provider may instruct the patient to physically locate herself in front of a particular background, or in a space free of objects, or including certain objects of known size, to enable calibration, greater depth perception and/or spatial awareness of the system. As one example, the system may instruct the patient to print out one or more visual calibration patterns and place it where it will be in view of the system. The system then transmits the guideline/instruction to the patient at step 315.

The patient may perform any steps (e.g. physical exam routine) or provide additional information as indicated by the guideline/instruction, and the patient's performance may be received by the speakers, cameras, keyboard, text entry, motion capture, and/or other sensors and is transmitted as feedback information to the system at step 320. Specifically, as the user interacts with the provider during the telemedicine encounter by participating in the musculoskeletal physical exam, the camera and other sensors capture video, audio, or other data and transmits the same as a feedback information signal to the system.

Generally, the feedback information may include a wide range of computer vision data (e.g., range of motion, wound inspection, and musculoskeletal kinematics) and may be audible and/or visual in nature. For example, audible feedback information may include auditory cues such as vocal expressions of pain or estimates of force applied by the user with a given set of contextual data, such as the weight of an object. Visual feedback information may include: calibration information, angular range of motion measurements (e.g., pauses in movements, collection of movements), wound healing assessment, kinematic alignment, and other clinical exam signs (e.g., instability and stiffness, appearance of soft tissues, etc.) In one embodiment, feedback information provided during the telemedicine encounter may be received in the form of video frames as a matrix of values based on pixels or the like, to the software application located either on the smart device, local computer, or a server.

At step 325, the system processes and stores the received feedback information and associates it with the current session. The system may interpret the feedback information through any number of various image recognition techniques, including, but not limited to, object detection, image segmentation, region-based segmentation, edge detection segmentation, segmentation based on clustering, etc. Specifically, the system may use such techniques to identify bodies, body parts, tissues, bruises, bumps, swelling and noises. The system may simultaneously assess for patient joint angles, ranges of motion, kinematics, velocity and spatial orientation. Patient measurements may be confirmed (e.g., by the system or the physician) based on comparison to a template where the system has registered baseline movements from the patient. The system may use visual calibration information to increase the accuracy of measurements based on feedback information. The system may further use the patient's template and physical examination maneuvers to determine key parameters for a subsequent physical examination (e.g., X is misaligned, Y is too slow, wound is still bleeding, etc.).

The system may interpret the feedback information in its processing of the information. Interpreted feedback information can also include interpretations of contextual data, which can, in one example, include auditory cues such as vocal expressions of pain or estimates of force applied by the user with a given set of contextual data, such as the weight of an object. In another embodiment, the interpreted data may include the performance of the user during a set of exam maneuvers, such as range of motion, and feedback provided to the user and viewer during and/or after the user performs a set of motions.

In certain embodiments, the system may additionally filter unwanted elements of the raw feedback information. Such filtering may include removing the noise and other irrelevant features carried by a received signal. In one embodiment, for example, a low-pass filter may be used to filter out the noise in the signal. In another embodiment, a moving average filter is used to filter out the noise in the raw signal. Other filters may be additionally or alternatively be used to increase the signal-to-noise ratio of the raw signal.

In certain embodiments, the system may classify the received feedback information (e.g., a movement performed by the user) based on one or more processed real-time signals, such as a matrix shift in pixel values. Classifying the physical exam maneuver performed by the patient helps the system identify and understand the movement being performed by the patient.

The software application may identify the patient feedback information, such as movement, being performed by the user, based on features present in one or more signals through standard computer vision techniques, including machine learning and other artificial intelligence-based processes. For example, the system may determine the physical exam maneuver being performed by the user based on one or more processed real time signals, based on pixel changes from the video capture by the camera and one or more sensors. The software may extract a set of statistical or morphological features present in a rolling window of one or more processed signals. Features may include amplitude, mean value, variance, standard deviation of the signal, the number of valleys and/or peaks, the order of valleys and/or peaks, the amplitude of valleys and/or peaks, the frequencies of valleys and/or peaks and/or the time period of valleys and/or peaks in one or more processed signals. For example, while performing shoulder abduction, the matrix of video frame pixels might record a single unique peak over a time period followed by a single valley over a time period. These extracted features may have to be converted to other contextual signals, such as a two-dimensional spatial frame of reference for which to derive an arc of motion. These signals are subsequently classified by the machine learning based algorithm to detect the movement and process the range of motion being performed by the user in real time. The system may also apply visual calibration techniques to the feedback information to correct for potential bias in incoming video information.

In one embodiment, the application may apply a template matching algorithm to identify unique or repetitive features (such as peaks and valleys in the signal) in a rolling window of one or more processed signals. The software may compare the features to a set of physical exam templates stored in a physical exam template database stored locally or remotely. Based on the comparison, the software may then select the exam templates in the database having features most similar to or most closely matching those present in one or more processed signals. Based on one or more or a combination of the selected physical exam templates, the software may identify and classify the movement being performed by the user from the aforementioned machine learning, or similar, artificial intelligence-based analytic processes. In another embodiment, the software may use machine learning algorithms to detect physical exam maneuvers, and/or classify or identify exam maneuvers performed by a user in a rolling window of one or more signals based on the repetitive or morphological features in one or more signals from video data derived from pixels or the alike.

The system may generally classify the feedback information using one or more classification and interpretation algorithms, such as machine learning algorithms, pattern recognition algorithms, template matching algorithms, statistical inference algorithms, and/or artificial intelligence algorithms that operate based on learning models. Examples of such algorithms include k-Nearest Neighbor (kNN), Naïve Bayes, Support Vector Machines (SVM), Artificial Neural Networks (ANN), and Decision Trees.

In one embodiment, after the application classifies the physical exam inspection or maneuver offered by the user, the application may quantify and characterize the movement being performed, such as range of motion, alignment or an appropriately healing wound.

The application may analyze and draw conclusions from the feedback information. For example, it may determine if range of motion is lacking or if a wound is healing poorly. In one embodiment, the application may determine such information based on the amplitude of the real peak relative to the whole signal, number of real peaks, and/or other contextual information. In another embodiment, the application may be determined by changes in the matrix of grayscale values representing video frames pixels, or the alike, with transitions along the sequences of matrices interpreted as any other relevant signal changes.

In another embodiment, the application may quantify other characteristics of the movement being performed by the user such as the speed or angle of the movement. The application may determine the time period over which the peak or valley or morphological feature in one or more signals from video frame matrix pixels occurs to determine the rate at which the user performs each exam maneuver. In one embodiment, the identifies groups of repeated movements performed by a user. The aforementioned algorithms subsequently analyze these process signals from the available camera and other sensors. For example, if a lower extremity exam is being performed and gait is analyzed, the mechanical axis based on the center of hip rotation and center of the ankle is first identified, potentially superimposed on the display, and classified from the user's movement as gait. The gait may then subsequently be analyzed as antalgic if the speed is diminished or if there exists mechanical malalignment or a varus thrust.

The application may also determine the physical exam maneuver being performed by the user based on one or more processed real-time signals, based on pixel changes from the video capture by the camera and one or more sensors. The software extracts a set of statistical or morphological features present in a rolling window of one or more processed signals. Features may include amplitude, mean value, variance, standard deviation of the signal, the number of valleys and/or peaks, the order of valleys and/or peaks, the amplitude of valleys and/or peaks, the frequencies of valleys and/or peaks and/or the time period of valleys and/or peaks in one or more processed signals. For example, while performing shoulder abduction, the matrix of video frame pixels might record a single unique peak over a time period followed by a single valley over a time period. These extracted features may have to be converted to other contextual signals, such as a two-dimensional spatial frame of reference for which to derive an arc of motion. These signals are subsequently classified by heuristics, background features, and/or machine learning algorithms to detect the movement and process the range of motion being performed by the user in real time.

In one embodiment, the application applies a template matching algorithm to identify unique or repetitive features (such as peaks and valleys in the signal) in a rolling window of one or more processed signals. The software compares the features to a set of physical exam templates stored in a physical exam template database stored locally or remotely. Based on the comparison, the software then selects the exam templates in the database having features most similar to or most closely matching those present in one or more processed signals. Based on one or more or a combination of the selected physical exam templates the software identifies and classifies the movement being performed by the user from the aforementioned machine learning, or similar, artificial intelligence-based analytic processes.

In one embodiment, the software determines the statistical characteristics such as a mean or standard deviation associated with the physical exam in one or more signals. For example, the software may determine the mean knee flexion from the amplitude of peaks recorded in one or more signals. If the mean is found to be relatively greater than that of the expected threshold, this may be displayed as a positive outcome attributed to the user's data profile. In another embodiment, the software uses machine learning algorithms to detect physical exam maneuvers, and/or classify or identify exam maneuvers performed by a user in a rolling window of one or more signals based on the repetitive or morphological features in one or more signals from video data derived from pixels or the alike.

At step 330, the system determines whether the current session is completed. If the current session is completed, then the system determines a treatment recommendation based on the session information and transmits the received information and recommendation to the provider at step 335. In certain embodiments, the system may, instead of automatically providing a recommendation to the patient, prompt the provider to prepare a recommendation.

In certain embodiments where the system determines the treatment recommendation, the system may first transmit the recommendation to the provider for review, and upon confirmation by the provider, the system may then transmit the treatment recommendation to the patient. In such embodiments, the system may adjust the algorithm for determining treatment recommendation based on the provider's feedback. In other embodiments, the system may automatically transmit the treatment recommendation to the provider and patient without requiring the provider to review and confirm the recommendation.

Treatment recommendations may be provided in the form of a report that may be visual, audible, or written and potentially displayed during the telemedicine encounter or a future encounter. The recommendation may be qualitative or quantitative in nature. Treatment recommendations may be delivered via video and/or written form (e.g., email, text message, social media, etc.). Treatment recommendations may also comprise instructions for future patient physical movements or therapeutic encounters, to be delivered via other providers (e.g. physical therapists) either in person or via a telemedicine encounter; or by a virtual physical therapist, animated onscreen for the patient.

In embodiments where the treatment recommendations are delivered in the form of a video (including an animation), the video may explain the proper form, or the provider may, in real time, provide direction verbally to the patient. The system may notify the patient or provider of a normal or abnormal provider examination finding and may display to the user a series of images or videos representing the desired examination goal. In one embodiment, the provider may receive physical exam data based on the exam performed by the patient. For example, the application may notify the patient and the provider if the patient is underperforming in the recovery process or not participating at all.

The system may also display additional information to the patient during and/or after the telemedicine encounter. For example, in one embodiment, it may overlay in real time the range of motion by the patient for both the patient and provider's reference during the telemedicine encounter.

If the current session is not completed, the system will update the current instruction to the next instruction/guideline and return to step 315.

In certain embodiments, the next instruction/guideline may also pertain to the initial inspection instruction/guideline. In other embodiments, it may pertain to an instruction/guideline pertaining to a physical examination. Generally, physical examination instruction/guidelines are transmitted upon the determination that the initial inspection has been completed.

The next instruction/guideline may be based on one or more of: patient information and parameters, feedback information, a predetermined template, provider input, etc.

During the physical examination, the patient may move their unaffected side first through entire range of motion with notation made for painful joint movement, joint range of motion, and dyskinesia. Patients may further be instructed to point to painful joint regions. Target body regions may include the entire spine, shoulder, elbow, wrist, hip, knee, and ankle. An example physical examination procedure in accordance with one or more embodiments is provided below:

### Physical Examination Procedure:

### Physical Examination Protocol and Parameters

### Upper Extremity Evaluation:

The upper extremity evaluation consists of examination of the shoulder, shoulder girdle, elbow and wrist. Upper extremity examination is performed according to the instructions below with the patient facing the camera directly (“front view”) and also in a side view. Shoulder, elbow and wrist kinematics are assessed using audiovisual capture and machine learning algorithms enabled as part of the telemedicine encounter.

### Shoulder Evaluation

Inspection
   Evaluation for bilateral symmetry with assessment for dyskinesia, gross deformity/displacement and loss of contour
Forward elevation
   Side view
   Instructions
      Stand with side facing camera. With your arm straight and elbow locked raise your arm straight over your head as high as possible. Once you get to the end hold for 2 seconds and return to starting position
   Normal
      160 degrees
Shoulder Abduction
   Front view
   Instructions
      Standing facing camera. Keep your arms straight and elbow locked. Bring your arm straight out to the side and continue until your arms are alongside your ears. Once you get to the end hold for 2 seconds and return to starting position.
   Normal
      160 degrees
Shoulder External rotation (0 degrees/arm at side)
   Front View
   Instructions
      Face camera with your arms at your side and elbow bent to 90 degrees. Rotate your hand out to the side keeping your elbow along your side. Once you get to the end hold for 2 seconds and return to starting position.
   Normal
      70 degrees
Shoulder internal Rotation (0 degrees/arm at side)
   Front View
   Instructions
      Facing camera with your arms at your side and elbow bent to 90 degrees. Rotate your hand in towards your stomach keeping your elbow along your side. Once you get to the end hold for 2 seconds and return to starting position.
   Normal 60 degrees
Shoulder Internal rotation reach behind back
   Front view (from behind)
   Instructions
      Stand with back facing camera. Take your hand and reach behind back as far as possible. Try and touch the opposite shoulder blade, sliding your hand up your back. Once you get to the end, hold for 2 seconds and return to starting position.
   Normal
      Able to touch bottom of opposite shoulder blade
Shoulder abduction with external rotation
   Side view
   Instructions
      Stand with your side facing the camera. Bring your elbows up to shoulder height and keep your elbows bent at 90 degrees. Your hand should be pointing directly in front of you. Once in this position take your arm and rotate it back as far as you can. Your hand should point toward the ceiling in normal circumstances. Once at the end, hold for 2 seconds and return to starting position.
   Normal
      80-90 degrees
Shoulder abduction with internal rotation
   Side view
   Instructions
      Stand with side facing camera. Bring your elbows up to shoulder height and keep your elbows bent at 90 degrees. Your hand should be pointing directly in front of you. Once in this position take your arm and rotate it forward/down as far as you can. Once at the end hold for 2 seconds and return to starting position.
   Normal
      40-50 degrees
Elbow Evaluation
Inspection
   In the frontal view, evaluate for bilateral symmetry, deformity and loss of contour
Elbow Carrying Angle
   Front View
   Instructions
      Stand facing the camera directly (frontal view). Arm at the side. Forearm at the side. Palms should be facing forward toward camera. Angle is measured at the elbow joint.
   Normal
      5-15 degrees
Elbow Flexion
   Side view
   Instructions
      Stand with side facing camera. Keeping your arm along your side and upper arm stationary and bend your elbow up as far as possible. Hold in this position for 2 seconds and return to starting position.
   Normal
      130 degrees
Elbow Extension
   Side view
   Instructions
      Stand with side facing camera. Keeping your arm along your side and upper arm stationary, straighten your arm as far as possible. Hold in this position for 2 seconds and return to starting position.
   Normal
      0 degrees

### Wrist Evaluation

Wrist Pronation
   Front view (elbow bent to 90 degrees)
   Instructions
      Stand facing the camera with elbow bent at 90 degrees. Keeping your hand open, turn your hand so that your palm faces the ground. Hold in this position for 2 seconds and return to starting position.
   Normal
      90 degrees
Wrist Supination
   Front view (elbow bent at 90 degrees)
   Instructions
      Stand facing camera and elbow bent at 90 degrees. Keeping your hand open, turn your hand so that your palm rotates towards the ceiling. Once you get to the end hold for 2 seconds and return to starting position.
   Normal
      80 degrees
Wrist Extension
   Side view
   Instruction
      Stand in side view of the camera. Place arm by your side. Place forearm at 90 degrees. Extend wrist maximally.
   Normal
      60 degrees
Wrist Flexion
   Side View
   Instruction
      Stand in side view of the camera. Place your arm at your side. Place your forearm at 90 degrees. Flex wrist maximally.
   Normal
      60 degrees

### Lower Extremity Evaluation:

Lower extremity evaluation consists of examination of the general lower extremity alignment, hip, knee and ankle. Evaluation is performed according to the instructions below with the patient facing the camera directly (“front view”) and also in “side view.” Hip, Knee and ankle kinematics are assessed using audiovisual capture and machine learning algorithms enabled as part of the telemedicine encounter.

### Lower Extremity Evaluation:

Inspection
   Patient starts with a front posture scan and a side posture scan
   Front Scan
      General Hip, knee, and ankle alignment
      Valgus/varus alignment based on mechanical axis assessment through the computed center of the knee
      Note will be made of the hip knee and ankle alignment (valgus, varus, neutral)
      Gait will be assessed as the patient walks towards and away from the electronic/audiovisual telemedicine capture device
   Side scan
      Recurvatum/Procurvatum
      Gait will be assessed as the patient walks to the left and right of the electronic/audiovisual telemedicine capture device

### Knee Evaluation

Inspection
   Frontal View
   Instructions
      Stand facing the camera. Angle is measured at the knee. Dropping a line from the hip joint to the knee, then to the center of the ankle joint in order to assess for the mechanical axis
   Normal
      3-6 degrees of valgus
Knee flexion/extension (supine)
   Side View
   Instructions
      Active range of motion (AROM)
         The patient will lay down face up on a bed, table or other flat surface. The camera will be adjusted so that the patient's side that is being measured is facing the camera. The patient starts with the knee as straight as possible (full extension AROM value). Then, while keeping the patient's heel on the bed/table, the knee is actively bent by the patient to the greatest degree of flexion possible (full flexion AROM value).
         If pain occurs during any part of the motion, the patient will notify the provider of painful range of motion.
         Repeat on unaffected side
      Passive range of motion (PROM)
         The patient will lay down face up on a bed, table or other flat surface. He will adjust the camera so that the side that is being measured is facing the camera. The patient will start with the knee as straight as possible. Then, while keeping the heel on the bed/table, the patient will bend the knee as much as possible, use manual assistance to provide some over-pressure, hold for 2 seconds (full flexion PROM value), and then return to the starting position
         If pain occurs during any part of the motion, the patient will indicate this to the examiner
         Repeat on unaffected side
   Normal Values
      0-140 degrees (AROM)
      0-150 degrees (PROM) “Heel to Glutes”
Knee flexion/extension standing (squatting)
   Side View
   Instructions
      The patient stands with side facing camera. The patient will start with knees as straight as possible (full extension AROM), and then the patient will squat down as much as possible, bending the knee in the process as much as range of motion allows before returning to the starting position.
      If pain occurs during any part of the motion, the patient will indicate the presence of pain
      Repeat on the unaffected side
      Range of motion parameters will be captured by computer-assisted and machine-learning models
   Front View
   Instructions
      The patient stands with their front facing camera. The patient will start with knees as straight as possible (full extension AROM). The patient will squat down as much as possible, bending the knee in the process as much as range of motion allows before returning to the starting position.
      Automated audiovisual capture will note if there is equal weight bearing on both feet, and if so, what percentage of deviation there is from midline
Knee flexion (single leg squat)
   Side View
   Instructions
      The patient will stand with their side facing camera. Standing on one leg, the patient will start with knee as straight as possible. The patient will then squat down as much as possible, bending the knee in the process as much as range of motion allows before returning to the starting position.
      If pain occurs during any part of the motion, the patient will notify the provider.
      Repeat on unaffected side
      Automated audiovisual capture will capture range of motion
   Front View
   Instructions
      The patient will stand with their front facing camera. While standing on one leg, the patient will stand with knee as straight as possible (full extension AROM). The patient will then squat down as much as possible, bending the knee in the process as much as range of motion allows before returning to the starting position
      If pain occurs during any part of the motion, the patient will notify the provider.
      Repeat on unaffected side.

### Hip Evaluation

Inspection
   On a frontal view, assessment will be made for boney prominences and symmetry of pelvis
   Patient will be asked to stand on one leg and then alternately stand on the other leg. Assessment will be made for dropped hip (“Trendelenburg Test”)
Point to spot of pain (Look for C-Sign)
   Front and side view
   Looking for Classic “C-Sign”
Hip Flexion Standing (Squatting)
   Side View Instructions
      Patient will stand with side facing camera. The patient will initially stand as tall as possible and then squat down as much as possible, and then return to the starting position.
      If pain occurs during any part of the motion, the patient will notify the provider.
      Repeat on unaffected side.
      Automated audiovisual capture will capture range of motion.
   Front View Instructions
      The patient will stand with their front facing the camera. The patient will initially stand as tall as possible and then squat down as much as possible, and then return to the starting position.
      Automated audiovisual capture will note range of motion and if there is equal weight bearing on both feet and if so what percentage of deviation there is from midline
Single leg squat
   Side View Instructions
      The patient will stand with their side facing the camera. While standing on one leg, the patient will start by standing as tall as possible. The patient will then squat down as much as possible, and then return to the starting position.
      If pain occurs during any part of the motion, the patient will notify the provider.
      Repeat on unaffected side.
      Automated audiovisual capture will capture range of motion.
   Front Instructions
      The patient will stand with their front facing the camera. While standing on one leg, the patient will start by standing as tall as possible. The patient will then squat down as much as possible, and then return to the starting position.
      If pain occurs during any part of the motion, the patient will notify the provider.
      Repeat on unaffected side.
      Automated audiovisual capture will capture hip, knee and ankle alignment (valgus, varus, neutral).
Single Leg Balance
   Front and Side view
      The patient will stand with their front facing the camera. While standing and facing camera, that patient will attempt to balance on one leg trying to maintain a “soft knee”. The patient will then perform this on both legs and then turn to perform this on a side view as well.
   Normal
      Able to maintain balance without hip dropping and a soft knee. (Slight knee flexion)
Seated internal rotation
   Front view only
      While seated on a bed, table or flat surface with feet hanging off (feet not touching the ground), the patient will actively rotate one foot away from the other as far as possible. Once you get to the end hold for 2 seconds and return to starting position.
      Repeat for the opposite extremity
   Normal
      25 degrees of Internal Rotation
Seated external rotation
   Front view only
      While seated on a bed, table or flat surface with feet hanging off (feet not touching the ground)—the patient will actively rotate one foot towards the opposite knee sliding up the shin. Once you get to the end hold for 2 seconds and return to starting position.
      Repeat for the opposite extremity
   Normal
      70 degrees of Internal Rotation
Supine Flexion
   Side view and front view
      While laying on the floor, bed, table or other flat surface the patient will actively bring the index knee to their chest. Once at the end range of motion, the patient will hold for 2 seconds and return to starting position. Repeat on opposite side.
   Normal
      110 degrees of motion
Side Lying Hip Abduction
   Front View
      While laying on floor, bed, table or other flat surface and facing camera, the patient will lift their heel towards the ceiling keeping their knee locked straight. The patient will note to the clinician if at any point there is pain.
   Normal
      60 degrees of pain free motion.

### Ankle Evaluation

Ankle Dorsiflexion/Plantar flexion
   Side View
   Instructions
      The patient will lay down face up on a bed, table or other flat surface. Care will be taken to adjust the camera so that the side of the patient that is being measured is facing the camera. The patient will start with the knee straight, and foot slightly off the edge of the bed or table. The patient will dorsiflex (move foot toward the head) the foot as much as possible and hold for 2 seconds (maximal dorsiflexion AROM). Patient will then plantarflex (move foot down like they are pushing down on the gas) their foot as much as possible for 2 seconds (maximal plantarflexion ROM). Patient will keep knee straight throughout the motion.
      If pain occurs during any part of the motion, the patient will notify the provider.
      Repeat on unaffected side.
   Normal Values
      Dorsiflexion—15 degrees downward from the foot's position from the ground
      Plantarflexion—50 degrees

### Spinal Evaluation

Inspection
   Frontal view
      The patient will front-face the camera and system will perform trunk alignment with assessment for scoliosis and lateral deviation from midline
   Side View
      The patient will side face the camera and the system will assess the patient for degree of cervical lordosis, thoracic kyphosis and lumbar lordosis. Assessment will also be made for forward head deviation and rounded shoulders.
Lumbar spine side bend/lateral bend
   Frontal View
   Instructions
      The patient will stand with the front facing the camera for a frontal view. Patient will bend at the waist maximally to the right and left sides. Angle is measured at waist. Zero degrees is a straight vertical line perpendicular to the floor. Angle is the displacement from zero of the line above the waist.
      Normal: 30-40 degrees
Lumbar spine flexion and extension
   Side View
   Instructions
      The patient will stand with side view towards the camera. The patient will bend at the waist maximally forward and backward. The patient will keep lower extremities straight. The angle will be measured at the waist. Zero degrees is a straight line, vertically, perpendicular to the floor. Angle is the displacement from zero of the line above the waist relative to the line below the waist.
   Normal
      Forward flexion: 65-75 degrees
      Extension: 25-35 degrees
Cervical Spine lateral bend
   Frontal view
   Instructions
      The patient will stand facing the camera for a frontal view. While keeping the shoulders stable, the patient will bring the head down laterally to the right and left sides (ears to the shoulders). Zero degrees is a straight line vertically drawn perpendicular to the floor. The angle is measured at the base of the cervical spine.
      Normal: 20-45 degrees
Cervical Spine Flexion and Extension
   Side View Instructions
      The patient will stand with the side view relative to camera. Patient will maximally bend the head forward and backward. Zero degrees is a straight line vertically drawn perpendicular to the floor. Angle is measured at the base of the cervical spine.
      Normal Flexion: 80 degrees
      Normal Extension: 60-70 degrees

At step 340, the system determines whether there are any additional sessions. If there are additional sessions, the system will update the current session to the next session and repeat the process at step 305. If there are no additional sessions, the method will end.

It will be appreciated that the system may also be used in non-examination mode. For example, in certain embodiments, the user may undergo physical therapy under observation, and the system may verify that the patient is doing the exercises correctly. The system may gather various reporting options for the feedback information and provide such information to physicians involved in the telemedicine encounter.

Referring to FIG. 4, a method 400 for facilitating automatic transcription of speech for a clinical note during a telemedicine encounter via artificial intelligence and/or machine learning algorithms is provided. In certain embodiments, the system may guide the patient through questions relating to the patient's medical history, symptoms, etc. Generally, as the patient interacts with the system during the telemedicine encounter, the speech recognition software performs both voice recognition and speaker identification while simultaneously capturing components of the speaker's speech and language. The system may then transcribe these components into a clinical note or other summative document. The provider may subsequently evaluate the patient and components of the provider's clinical assessment is included in the same medical transcript in order to provide a complete clinical note or other summation.

At optional step 405, the system displays a question. In other embodiments, the physician may instead conduct the questioning.

At step 410, the system receives a response to the question from the patient via one or more sensors (e.g., speakers). The response may generally be in the form of audio data. The audio data, including sound waves, pronunciation, speech, and cadence, is registered by the software application.

At step 415, the system generates a transcript from the response. Generally, the system inspects and tracks the sound waves and auditory components of the audio data received. The software application conducts speech recognition and speaker recognition analysis on this audio data using acoustic and language modeling algorithms available in machine learning and other artificial intelligence-based processes. Automated speech recognition algorithms may include deep learning frameworks such as connectionist temporal classification, sequence-to-sequence and online sequence-to-sequence. In the preferred embodiment, neural networks are utilized to provide a framework for acoustic and language models. In certain embodiments, speech prediction techniques such as the Generative Pre-Trained Transformer version 3 or 4 (GPT-3 or GPT-4), developed by OpenAI (See https://openai.com) may be used to improve transcription accuracy, correct errors in speech transcription or classification, or propose responses to patient statements or questions.

As an example, to provide fidelity, the application may compare the provider and patient speech pattern to a baseline reference obtained from each individual telemedicine encounter participant, or the application can compare collected auditory data to a repository of normative acoustic and language references.

It will be appreciated that the provider may engage in discussion with the patient (e.g., to ask follow-up questions), and the provider's speech may similarly be captured and transcribed by the system. For example, the system may be trained to identify and recognize specific features of the provider's speech and language such that the medical transcription is derived primarily from the provider's auditory input during the telemedicine encounter. In such embodiments, the application may, without prompting, transcribe speech based on the provider's data input elements of a clinical note or medical transcription. The application may further fill out one or more elements of other forms or database tables by detecting the appropriate use, category, or destination of various language elements.

In other embodiments, the application may, instead of transcribing auditory information captured during the telemedicine encounter, cue the provider to provide verbal responses including medical dictation or pertinent parts of a clinical note which can then be transcribed using the techniques described herein such as deep learning machine-learning algorithms, or any other process rooted in computer speech recognition and artificial intelligence.

At step 420, the system transmits the transcript from the recorded response. The transcription may be organized according to a preformatted template or may be provided as a raw data output. The transcript will generally be transmitted to the provider and will allow for the provider to review and edit the medical transcription as appropriate. In other embodiments, the transcript may be transmitted to the patient as well. In yet other embodiments, the transcribed text may be routed to a third party for correction prior to transmittal to the provider.

At step 425, the system receives transcription feedback information from the provider. Transcription feedback information generally consists of feedback regarding the accuracy of a transcript generated by the system. Such transcription feedback information may be, for example, in the form of a score (e.g., on a scale of 1 to 100 with 1 indicating the lowest accuracy and 100 indicating the highest accuracy). The transcription feedback information may additionally or alternatively consist of text indicating the level of accuracy and the reason for the indication. Although not shown, the system may automatically update the transcript according to the transcription feedback information. In other embodiments, the system may additionally or alternatively allow the provider to update the transcript manually. In some embodiments, the system may transmit recorded responses and transcripts to other individuals or 3^{rd} parties for transcript checking or to obtain additional transcription feedback information.

At step 430, the system updates speech and speaker recognition algorithm based on the received transcript feedback information.

Referring to FIG. 5, a method 500 for dynamically pricing a telemedicine appointment is provided. Generally, dynamic pricing may be utilized in order to create an intelligent system that prices physician virtual services in real time based on a set of algorithms that is customized to each individual patient-physician encounter. As part of the physician selection process, the patient interacts with the system in order to provide input that is used to calibrate the software's dynamic price points. As part of the software system and service delivery, the patient or similar patient representative may pay for the virtual encounter outside of the traditional insurance-based payment system, or the encounter may be paid for using insurance or government payor based systems.

At step 505, the system stores provider information associated with one or more providers. Provider information may include: reputation information (e.g., ratings, reviews, years in practice, residency program, fellowship program, etc.), specialty, education information (e.g., undergraduate school, medical school, etc.), scheduling information (e.g., available times for appointments), location of practice, etc.

At step 510, the system receives patient preferences information associated with a patient who requests an on-demand telemedicine appointment. The system may further receive patient information (e.g., if a patient is using the system for the first time).

Patient preferences information may include budget for virtual encounter, preferred location of the encounter, preferred provider characteristics (e.g., gender, years of practice, medical school, undergraduate school, specialty, other skill or ability information, etc.) It may further include a ranking indicating the level of importance of each preference (e.g., budgetary concerns may have a top ranking indicating a high level of importance; proximity may have a low ranking indicating a low level of importance). For example, the ranking system may be from a scale of 1 to 5, with 1 indicating the lowest level of importance, and 5 indicating the highest level of importance.

In such embodiment, for example, the system may ask the patient to rank the importance of physician pricing and fee structure to their selection process and eventual choice of service provider for a virtual encounter. If a patient indicates that they do not have significant price sensitivity, then physician services across all price points may be shown to the patient. If patient indicates price sensitivity, then further questions around patient willingness are asked in order to tailor provider recommendations to the patient price point.

In one embodiment, patient preferences information may be automatically determined by the system. For example, patient willingness to pay and price sensitivity may be obtained passively from general patient characteristics and available online information. Patient characteristics representative of willingness to pay can be derived from information sources such as patient zip code, patient self-reported income, credit card type, credit score, occupation, purchasing/spending habits, websites visited and online memberships. For example, a low credit score may indicate that a patient has low willingness to pay/high price sensitivity. These characteristics may then be used as a proxy for patient income, and targeted recommendations can be made based on patient willingness to pay and a matched set of physicians that are within the patient's willingness to pay.

At step 515, the system determines one or more matching providers, available appointment times, and optimum pricing based on patient preferences information, provider information, and one or more industry factors.

In certain embodiments, patient willingness to pay can be captured in proxy fashion as outlined above based on patient characteristics. In such embodiment, provider pricing for requested virtual encounters may be adjusted in a dynamic pricing in order to reflect a fair rate based on patient's income strata. Thus, a patient with high disposable income may be charged a higher fee in order to subsidize a patient with limited disposable income who can then be charged a lower fec.

The dynamic pricing model for a virtual physician encounter may be adjusted in order to reflect the anticipated complexity of services needed to fulfill a comprehensive clinical encounter. For example, in certain embodiments, a low-severity condition may be ascertained based on software inputs that are either passively obtained or directly inputted from either the patient or physician. Physician fees may then be dynamically priced to reflect the low-severity condition (i.e. through a lower price point). In similar fashion, a complex condition that requires an intensive encounter or high level of physician involvement may be dynamically priced at a premium to reflect the high complexity of the case.

In one embodiment, the dynamic pricing model is adjusted based on the supply of and demand for providers associated with the platform and physician network. For example, in certain situations, based on the parameters provided by the patient, there may be a paucity of available providers to complete the virtual episode. In the scenario where there are a limited number of physicians meeting the patient's criteria, premium pricing can be applied in order to account for the limited supply and increased demand, or to encourage additional providers to provide additional telemedicine encounter openings. Similarly, an iteration of the model can allow for diminution of physician fees if there is an excess supply of physician services with limited demand for said services.

Dynamic pricing may also be partially dictated by the physician based on physician availabilities represented in a synced physician calendar. In certain embodiments, for example, physicians can delineate times during which they are available for a virtual consultation. The physician can also delineate times where they wish to be available but at a premium price. As part of this algorithm, the software can seek to optimize the patient's willingness to pay with the physician's willingness to provide service. In certain embodiments, physicians who would otherwise be unwilling to engage in a virtual encounter may be solicited at a price point that would motivate them to participate in the virtual encounter. Calendar management can include considerations for time of day as well as well as day of the week. For example, premium hours may include late evenings and weekends while non-premium hours may include weekday business hours.

In certain embodiments, dynamic pricing may be applied for premium services designated to the patient as warranting premium pricing. In this embodiment, physicians with desirable characteristics are subject to dynamic pricing and increased price attribution based on these desirable characteristics. Desirable characteristics may include high patient ratings, high reputation scores, in-depth experience, or simply physicians assigned by the system software (or software administrators) as premium.

In certain embodiments, dynamic pricing for services may additionally or alternatively be set by patients. In this model a patient requests a telemedicine appointment through the platform and software. The patient lists their willingness to pay a certain price. The patient and patient's clinical needs may then be presented to the physician in auction format with physicians bidding for the telemedicine appointment. The physician can either accept the encounter at the posted price or offer a higher price. If no physician accepts the price at the stated patient price, then the patient may be presented with a list of physician bids that are proximate to but higher than the patient price point. At this point the patient can select the desired physician.

Auction-style dynamic pricing may additionally or alternatively be employed with physicians setting their desired price for a telemedicine appointment. In one example of this model, patients are presented with the physician's characteristics such as reputation, years of experience and specialty areas. The patients then bid on the physician's service within a finite period of time. At the end of the allotted time, the physician's service is provided to the highest bidding patient. If no bids are made, the physician and software have the option to re-post physician's service at the same or lower rate. Other variations on an auction-style pricing model will be apparent to the reader and are within this disclosure.

At step 520, the system transmits, to the patient, provider information associated with one or more matching providers, available appointment times, and determined prices associated with the providers.

At step 525, the system receives a selected provider and appointment time.

At step 530, the system optionally transmits a request to confirm the appointment time to the provider. The request generally includes at least a portion of the patient information and the requested appointment time. Although not shown, in certain embodiments, if the appointment time is declined by the provider, the system may require the provider to provide a reason for declining the appointment. The system may additionally or alternatively penalize the provider for declining appointments (e.g., by ranking the provider lower in its algorithm such that the provider is shown on a less frequent basis to patients). On the other hand, the system may reward providers for consistently accepting appointments (e.g., by weighting the provider higher in its algorithms, by featuring the provider on the application, making a note of the provider's consistency on the provider's profile, paying a bonus to the provider, providing online shopping discounts to the provider, etc.)

At step 535, the system optionally receives the confirmation from the provider to book the appointment and appointment time.

At step 540, the system transmits the confirmation including at least a portion of the provider information and appointment time to the patient.

Referring to FIG. 6, an exemplary matched provider screen 600 illustrating the provider-tiered pricing structure in one embodiment is provided.

As discussed above, upon a patient's submission of patient preference information, the system determines and transmits provider information associated with one or more matching providers to the patient. In embodiments with more than one matching provider, the system may rank the providers in order of percent match, price, etc. As shown, the screen may display a portion of the provider information, including: physician name and title(s) 605 (e.g., Benedict Nwachukwu, MD, MBA), photo, undergraduate college attended 610 (e.g., Columbia University), medical school attended 615 (e.g., Harvard University), hospital where training was completed 620 (e.g., Hospital for Special Surgery), current affiliated hospital 625 (e.g., Hospital for Special Surgery—Cornell Medical College), specialty 630 (e.g., hip & knee replacement), sub-specialty, etc. The screen may further include a link allowing the patient to learn more about the provider 645 (e.g., years of experience, ratings, testimonials, research, consistency in accepting appointments, response time, etc.).

The screen may also include one or more links 635, 640 associated with each provider with the provider's next available times for an appointment. The screen may also include a link 655 allowing the patient to view all available times for the provider. In the embodiment shown, once a link 640 is selected, it may be displayed in a different color than the other links (e.g., black) to indicate that it is currently selected. The screen may also display one or more prices 650, 660, 665 associated with each provider. In the shown embodiment, three different providers at three different price points, least cost (e.g., $450), average cost (e.g., $580) and highest cost (e.g., $800) are displayed. It will be appreciated that in certain embodiments, the system may display all of the different price points possible as a result of a specified patient preference (e.g., the patient has indicated high price sensitivity), while in other embodiments, the system may only display certain price points (e.g., the system may display only the highest cost option if the patient has indicated low price sensitivity.)

Referring to FIG. 7, a provider availability screen 700 according to an embodiment is illustrated. Such screen may show, in addition to the name and title 705, location 710 of the provider and price 715 of the appointment, a detailed calendar 720 of the provider's full schedule, including available appointment times 725, unavailable appointment times 730, whether the appointment is priced at a regular or premium price 735, one or more selected appointment times 740, etc. Unavailable appointment times, available appointment times, premium appointment times, and selected appointment times may each be displayed in different colors. For example, unavailable appointment times may be indicated in gray, available appointment times may be indicated in green, premium appointment times may be indicated in yellow, and selected appointment times may be indicated in blue. The screen may also allow the patient to navigate from the current physician availability screen 700 to the “Overview” screen (e.g., by selecting the “Overview” tab 745).

Referring to FIG. 8, a method for matching a patient to a physician for an on-demand telemedicine encounter is provided. In one embodiment, the patient completes a clinical intake providing patient information (e.g., medical history). This patient information can then be used to match the patient with the closest matching physician(s) based on pre-populated provider proficiencies. Once the system receives a selection of one or more matching physicians, the system may charge the consultation fee to the patient and immediately thereafter notify the physician(s) of the booking in order of the patient's preferred physician(s). The encounter is booked and confirmed once a selected physician confirms the availability and willingness to perform the consultation.

At step 805, the system stores provider information associated with one or more providers.

At step 810, the system receives, from a patient, a request for an on-demand virtual encounter including patient information associated with the patient. Generally, on-demand virtual encounters may include scheduling immediately and up to 48 hours into the future.

In one embodiment, the request for the on-demand virtual encounter may include a selection indicating the urgency of the virtual encounter. In such embodiment, a request for immediate physician access may need scheduling within the next three hours, while less immediate access may allow for a window of 24 hours to schedule the appointment. The immediacy of the patient's telemedicine appointment need may then be matched with a physician's availability calendar.

At step 815, the system receives patient preference information. In certain embodiments, the system may also receive a ranking of one or more categories of the received patient preference information. For example, various provider characteristics may be ranked a scale of importance (e.g., 1 to 5 with 5 being the highest rank) with a goal for the platform to optimize the categories marked by the patient as most important.

At step 820, the system determines one or more matching providers based on the patient preference information and provider availability. The system may also determine the matching providers based on provider information.

At step 825, the system transmits at least a portion of provider information associated with the matching physician(s) to the patient. Such displayed provider information includes at least next available appointment times and prices associated with each matching provider.

At step 830, the system receives a selection of one or more providers, a preference ranking associated with each provider (e.g., based on order of likeability and preference), and a desired appointment time associated with each matching physician. In such embodiments, a patient may designate his first-choice provider with a first preference ranking, the second provider with a second preference ranking, and so on.

At step 835, the system determines and transmits a consultation fee charge to the patient. As discussed above with respect to FIG. 5, the consultation fee charge may be dynamically priced according to one or more factors including: patient preferences, physician availability, physician characteristics, industry factors, etc. Generally, since the consultation charge associated with each physician may vary, in certain embodiments, it may be determined based on price of the appointment with the first provider with the first preference ranking. In such embodiments, if the first provider is unable to make the appointment and a second provider with a lower or higher consultation charge is instead confirmed, then the system may send the patient a second charge if the final consultation fee charge is higher or refund/provide a credit for the difference if the final consultation fee charge is lower.

In other embodiments, it will be appreciated that the initial consultation charge may instead, be the lowest and/or highest charge determined based on the selected providers and/or appointment times. Again, in such embodiments, depending on the final booked appointment price, then the system may send the patient a second charge if the final consultation fee charge is higher or refund/provide a credit for the difference if the final consultation fee charge is lower.

At step 840, upon receipt of the consultation fee, the system transmits a notification (e.g., via email, text, phone call) to the provider associated with the first preference ranking. The notification may include patient information, the selected appointment time, and the price associated with the selected appointment time. In certain embodiments, the provider is contacted first via an initial text message. The provider may respond affirmatively to the text message prompt in order to confirm the requested encounter.

The system may receive the confirmation from the provider at step 845. Although not shown, the system may provide a reward (e.g., ranking the provider higher in its algorithms) to the provider for confirming the appointment time, for responding within a certain amount of time, etc. The system may further incentivize providers (through, e.g. monetary rewards or penalties) based on other metrics such as response times, frequency of accepting appointments or new patients, or accepting certain types of appointments depending on demand for those appointment types.

At step 850, upon receipt of a confirmation from the provider, the system transmits at least a portion of the confirmed appointment information (e.g., appointment time, physician name, length of appointment, etc.) to the patient.

If, instead, after a predetermined period of time (e.g., another 15 minutes), the system does not receive a response from the physician associated with the first ranking, the system may transmit another notification to the provider with a similar prompt for the provider to accept the requested encounter at step 855. Although not shown, if no action has been taken after the initial series of text messages, an automated phone call may then be generated by the system with a call to action (accept consultation) once the provider picks up the call.

If the confirmation is received from the provider at step 860, then the system returns to step 850 and ends. If, however, still no confirmation is received from the provider at step 860, then the system determines whether the patient has selected any other providers at step 865. If the system determines that additional providers were selected, then it will update the provider to the next selected provider associated with the next preference ranking at step 875 and repeat the process at step 840. If the system determines there are no additional selected providers, it will transmit a refund to the patient at 870, including a notification that it was unable to schedule the appointment.

Referring to FIG. 9, a provider availability screen 900 according to an alternative embodiment is illustrated. Similarly to FIG. 7, the screen displays the provider name (e.g., “Sonia Florian”), address, (e.g., “2100 Erwin Rd., Durham, NC 27705, USA”), total price of the encounter, and one or more provider availabilities that a patient may select. Additionally, the screen may display the earliest available time of the provider 905 (e.g., “Jan. 20, 2021 at 7:00 PM”), availability over the next three hours 920, and additional available times over the next 48 hours 910 (e.g., “next 3 hours: (7:00 PM-7:30 PM)”). In the embodiment shown, the additional availability over the next 48 hours may be displayed in the form of a drop-down box 910 showing the first availability 915 within the next 48 hours. The patient may select the drop-down box to view additional provider availabilities over the next 48 hours.

In some embodiments, the system may configured to offer partial or full “white label” or unbranded telemedicine platforms, either to other third parties or to individual physicians. The system may be configured to offer a provider “home page” at either a specified path on the system's main url (e.g. https://bicmd.com/DOCTOR_OATH) or at an independent URL selected by a provider (e.g. https://DOCTOR_URL/). The system may permit the provider to request a provider home page entirely through the system, through back-end API driven steps including provisioning the URL path, purchasing and setting up a domain name using a third party service such as Amazon Route 53. The system may allow the provider to partially or fully configure their home page using commonly available web-page building tools, or by uploading customized HTML or other website design files. The system may allow any information or features described herein, or commonly used on other web sites or web-based systems presently known or yet to be discovered, to be provided on a provider's home page. For example, a provider home page may incorporate information about the provider's education, training, or experience (see FIG. 6), availability (see FIG. 6-7), provide certain physical exam routines or exercise instructions (see FIG. 3), or even as questions of a prospective patient, the responses to which could be transcribed and transmitted to the provider (see FIG. 4). A provider home page could be branded (e.g. with the trademark or other identifying information of the system provider) or alternately could be made to appear as if it was solely associated with the physician herself.

Reference in the specification to “one embodiment” or to “an embodiment” means that a particular feature, structure, or characteristic described in connection with the embodiment is included in at least one embodiment. The appearances of the phrase “in one embodiment” or “an embodiment” in various places in the specification are not necessarily all referring to the same embodiment.

Unless specifically stated otherwise as apparent from the following discussion, it is appreciated that throughout the description, discussions utilizing terms such as “processing” or “computing” or “calculating” or “determining” or “displaying” or “determining” or the life, refer to the action and processes of a computer system, or similar electronic computing device (such as a specific computing machine), That manipulates and transforms data represented as physical (electronic) quantities within the computing system memories or registers or other such information storage, transmission or display devices.

Certain aspects of the embodiments include process steps and instructions herein in the form of an algorithm. It should be noted that the process steps and instructions of the embodiments can be embodied in software, firmware or hardware, and when embodied in software, firmware or hardware, and when embodied in software could be downloaded to resided on and be operated from different platforms used by a variety of operating systems. The embodiments can also be in a computer program product, which can be executed on a computing system.

The embodiments also relate to an apparatus for performing the operations herein. This apparatus may be specially constructed for the purposes, e.g. a specific computer, or it may comprise a general-purpose computer selectively activated or reconfigured by a computer program stored in the computer. Such a computer program may be stored in a computer readable storage medium, such as but is not limited to, any type of disk including floppy disks, optical disks, CD-ROMs, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Memory can include any of the above and/or other devices that can store information/data/programs and can be transient or non-transient medium, where a non-transient or non-transitory medium can include memory/storage that stores information for more than a minimal duration. Furthermore, the computers referred to in the specifications may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient to construct more specialized apparatus to perform the method steps. The structure for a variety of these systems will appear from the description herein. In addition, the embodiments are not described with reference to any particular programming language. It will be appreciated that a variety of programming languages may be used to implement the teachings of the embodiments as described herein, and any references herein to specific languages are provided for disclosure of enablement and best mode. While particular embodiments and applications have been illustrated and described herein, it is to be understood that the embodiments are not limited to the precise construction and components disclosed herein and that various modifications, changes, and variations may be made in the arrangement, operation, and details of the methods and apparatuses of the embodiments without departing from the spirit and scope of the embodiments as defined in the appended claims.

Various embodiments are described in this specification, with reference to the detailed discussed above, the accompanying drawings, and the claims. Numerous specific details are described to provide a thorough understanding of various embodiments. However, in certain instances, well-known or conventional details are not described in order to provide a concise discussion. The figures are not necessarily to scale, and some features may be exaggerated or minimized to show details of particular components. Therefore, specific structural and functional details disclosed herein are not to be interpreted as limiting, but merely as a basis for the claims and as a representative basis for teaching one skilled in the art to variously employ the embodiments.

The embodiments described and claimed herein and drawings are illustrative and are not to be construed as limiting the embodiments. The subject matter of this specification is not to be limited in scope by the specific examples, as these examples are intended as illustrations of several aspects of the embodiments. Any equivalent examples are intended to be within the scope of the specification. Indeed, various modifications of the disclosed embodiments in addition to those shown and described herein will become apparent to those skilled in the art, and such modifications are also intended to fall within the scope of the appended claims.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

All references, including patents, patent applications and publications cited herein are incorporated herein by reference in their entirety and for all purposes to the same extent as if each individual publication or patent or patent application was specifically and individually indicated.

What is claimed is:

1. A computer-implemented method comprising:
storing provider information associated with one or more providers;
receiving a request for a telemedicine appointment from a patient, the request comprising:
patient information and patient preferences information associated with the patient,
wherein the patient preferences information comprises:
a budget range for the telemedicine appointment,
a preference ranking associated with the budget range,
a location of the telemedicine encounter, and
a preference ranking associated with location;
determining, based on one or more of patient information, the provider information, and the patient preferences information, a matching provider and a fee associated with the matching provider;
transmitting matching provider information associated with the matching provider to the patient,
wherein the matching provider information comprises a plurality of available appointment times and the fee;
receiving a selection of the matching provider and an available appointment time;
transmitting the fee to the patient;
receiving the fee; and
transmitting a confirmation to the patient,
wherein the confirmation comprises the name of the selected matching provider, the selected appointment time, and the received fee.

2. A computer-implemented method comprising:
storing provider information associated with one or more providers;
receiving a request for an on-demand telemedicine appointment from a patient, the request comprising patient information and patient preferences information associated with the patient,
wherein the patient preferences information comprises an indication of the degree of urgency of the request and a location;
determining, based on the patient preferences information and the provider information, a plurality of matching providers;
transmitting, to the patient:
at least a portion of the provider information associated with each of the plurality of matching providers,
wherein the provider information comprises one or more available appointment times and a location;
receiving a selection of two providers from the plurality of matching providers,
wherein the first matching provider is associated with a first preference ranking; and
wherein the second matching provider is associated with a second preference ranking transmitting a fee to the patient,
wherein the fee is associated with the first matching provider;
receiving the fee;
transmitting a notification to the first matching provider, wherein the notification comprises:
appointment information comprising patient information and the appointment time, and
a request to confirm the appointment time;
determining that the first matching provider is not available;
transmitting a notification to the second matching provider, wherein the notification comprises:
appointment information comprising patient information and the appointment time, and
a request to confirm the appointment time;
receiving a confirmation, by the second matching provider, to the notification;
transmitting the confirmation to the patient,
wherein the confirmation comprises the name of the second matching provider, the appointment time, and the received fee.
